(19) United States
(12) Patent Application Publication
(10) Pub. No.: US 2017/0285014 A1
(43) Pub. Date: Oct. 5, 2017

(54) METHODS FOR THE DETECTION OF CYANIDE BASED ON DISPLACEMENT OF THE GLUTATHIONE LIGAND OF GLUTATHIONYLCOBALAMIN BY CYANIDE

(71) Applicant: RESEARCH COOPERATION FOUNDATION OF YEUNGNAM UNIVERSITY, Gyeongsan-si (KR)

(72) Inventor: Ji-Hoe KIM, Suseong-gu (KR)

(73) Assignee: RESEARCH COOPERATION FOUNDATION OF YEUNGNAM UNIVERSITY, Gyeongsan-si (KR)

(21) Appl. No.: 15/250,560

(22) Filed: Aug. 29, 2016

(30) Foreign Application Priority Data
Mar. 29, 2016 (KR) 102016-0037410

(51) Int. Cl. G01N 33/52; G01N 21/78; G01N 21/64; G01N 21/33

(52) U.S. Cl. CPC G01N 33/52; G01N 21/33; G01N 21/78; G01N 21/6428; G01N 21/643

(57) ABSTRACT

Provided is a method for the detection of cyanide based on displacement of the glutathione ligand of glutathionylcobalamin by cyanide. The composition for detecting cyanide (CN⁻) including glutathionylcobalamin (GSCbl) and a buffer has specificity in which GSCbl does not react with other anions by nucleophilic substitution, but selectively reacts with only CN⁻ by displacement. Further, the GSH bound to the Cbl reacts with CN⁻ by nucleophilic substitution with high efficiency to enhance sensitivity, and cyanocobalamin (CNCbl), di-cyanocobalamin (diCNCbl) and glutathione (GSH) which are byproducts generated by nucleophilic substitution reaction of CN⁻ may be qualitatively/quantitatively detected through spectrophotometric, naked eye, and fluorometric assays, respectively.

### CROSS-REFERENCE TO RELATED APPLICATIONS

This application is based on and claims priority from Korean Patent Application No. 10-2016-0037410, filed on Mar. 29, 2016 with the Korean Intellectual Property Office, the disclosure of which is incorporated herein in its entirety by reference.

### TECHNICAL FIELD

The present disclosure relates to a method for the detection of cyanide based on displacement of the glutathione ligand of glutathionylcobalamin by cyanide.

### BACKGROUND

Recently, as it is recognized that anions play a chemically or biologically important role, in a supramolecular chemistry field, it is very interested in developing a system of recognizing the anions. Cyanide anion is highly toxic and causes vomiting loss of consciousness and death, and thus it is particularly important to develop a system for detecting cyanide anion.

Cyanide anion is an important compound used in various industrial processes including gold mining, electroplating, and metallurgy, but the sudden release of the cyanide anion may cause a very serious problem. In this aspect, various systems for detecting cyanide anion have been continuously studied.

Many cyanide detection systems were developed using fluorescence resonance energy transfer, nucleophilic addition reaction, and de-metallization of metal chelates. However, most of cyanide detection systems are complex or show insufficient selectivity and sensitivity of the cyanide detection.

Accordingly, a simple method with high sensitivity and selectivity is required for the detection of cyanide.

Glutathionylcobalamin (GSCbl) is one of vitamin B12 derivatives, in which glutathione (GSH) is bound to the central cobalt (Co) of cobalamin (Cbl) in the upper axial position.

The inventors of the present disclosure found that the glutathione (GSH) ligand of glutathionylcobalamin (GSCbl) is displaced by cyanide by a nucleophilic reaction, which was applied to the cyanide anion detection.

Particularly, the inventors found that the composition for detecting cyanide (CN⁻) including glutathionylcobalamin (GSCbl) and a buffer according to the present disclosure has specificity in which GSCbl does not react with other anions by nucleophilic substitution, but selectively reacts with only CN⁻ by displacement, and further, GSH bound to Cbl reacts with CN⁻ by nucleophilic substitution with high efficiency to enhance sensitivity, and cyanocobalamin (CNCbl), di-cyanocobalamin (diCNCbl) and glutathione (GSH) which are byproducts generated by nucleophilic substitution reaction of CN⁻ may be qualitatively/quantitatively detected through spectrophotometric, naked eye, and fluorometric assays, respectively. As a result, the inventors completed the present disclosure.

### RELATED ART DOCUMENT

### Patent Document

### (Patent Document 1) Korean Registered Patent No. 10-1007847

### SUMMARY

The present disclosure has been made in an effort to provide a composition for detecting cyanide (CN⁻) including glutathionylcobalamin (GSCbl) and a buffer.

Further, the present disclosure has been made in an effort to provide a spectrophotometric detection method of cyanide using the composition.

Further, the present disclosure has been made in an effort to provide a naked eye detection method of cyanide using the composition.

Further, the present disclosure has been made in an effort to provide a kit for detecting cyanide (CN⁻) from a blood or food sample including the composition.

Further, the present disclosure has been made in an effort to provide a fluorometric detection method of cyanide using the composition.

An exemplary embodiment of the present disclosure provides a composition for detecting cyanide (CN⁻) including glutathionylcobalamin (GSCbl) and a buffer.

Another exemplary embodiment of the present disclosure provides a spectrophotometric detection method of cyanide, comprising the steps of: adding a sample including cyanide (CN⁻) to a buffer including glutathionylcobalamin (GSCbl) (Step 1);

reacting the cyanide (CN⁻) added in Step 1 with GSH by displacement through nucleophilic attack, in which the GSCbl has a characteristic that glutathione (GSH) is bound to cobalt (Co) which is a central metal of cobalamin (Cbl) having a planar structure in an axial direction (Step 2); and

qualitatively or quantitatively detecting the CNCbl generated in Step 2 by a UV-Vis spectrophotometer (Step 3).

In the spectrophotometric detection method, the buffer of Step 1 may have pH lower than pH 9.

Furthermore, yet another exemplary embodiment of the present disclosure provides a naked eye detection method of cyanide, comprising the steps of: adding a sample including cyanide (CN⁻) to a buffer including glutathionylcobalamin (GSCbl) (Step 1); reacting the cyanide (CN⁻) added in Step 1 with GSH by displacement through nucleophilic attack, in which GSCbl has a characteristic that glutathione (GSH) is bound to the central cobalt (Co) of cobalamin (Cbl) having a planar structure in an axial direction (Step 2); and detecting the diCNCbl generated in Step 2 by the naked eye by a change in color (Step 3).

In the naked eye detection method, the buffer of Step 1 may have pH 9 or lower pH.

Further, still another exemplary embodiment of the present disclosure provides a kit for detecting cyanide (CN⁻) in water, blood or food samples.

Furthermore, still yet another exemplary embodiment of the present disclosure provides a fluorometric detection method of cyanide, comprising the steps of: adding a sample including cyanide (CN⁻) to a buffer including glutathionylcobalamin (GSCbl) (Step 1);

in which GSCbl has a characteristic that glutathione (GSH) is bound to the central cobalt (Co) of cobalamin (Cbl) having a planar structure in an axial direction; reacting the cyanide (CN⁻) added in Step 1 with GSH by displacement through nucleophilic attack (Step 2); and

qualitatively or quantitatively detecting the GSH generated in Step 2 by a fluorometric detector (Step 3).

According to the exemplary embodiments of the present disclosure, the composition for detecting cyanide (CN⁻) including glutathionylcobalamin (GSCbl) and a buffer has specificity in which GSCbl does not react with other anions by nucleophilic substitution, but selectively reacts with only CN⁻ by displacement. Further, GSH bound to Cbl reacts with CN⁻ by nucleophilic substitution with high efficiency to enhance sensitivity, and cyanocobalamin (CNCbl), di-cyanocobalamin (diCNCbl) and glutathione (GSH) which are byproducts generated by nucleophilic substitution reaction of CN⁻ may be qualitatively/quantitatively detected through spectrophotometric, naked eye, and fluorometric assays, respectively.

The foregoing summary is illustrative only and is not intended to be in any way limiting. In addition to the illustrative aspects, embodiments, and characteristics described above, further aspects, embodiments, and characteristics will become apparent by reference to the drawings and the following detailed description.

### BRIEF DESCRIPTION OF THE DRAWINGS

FIGS. 1A-1D show a reaction absorption spectrum of 20 μM GSCbl and 1 mM KCN in 50 min Tris/HCl (pH 7.5). CNCbl is generated via the intermediate diCNCbl in the reaction of excess cyanide with GSCbl.

FIG. 1A is an absorption spectrum of GSCbl (dotted line) and a reaction (solid line) immediately after a start of the reaction.

FIG. 1B is an absorption spectrum measured every 5 minute of the reaction after FIG. 1A. An arrow represents an increase and a reduction of absorption according to a reaction.

FIG. 1C is a graph illustrating a change in absorption at a wavelength indicated according to a reaction time.

FIG. 1D is a graph illustrating a comparison of absorption spectrums of the indicated cobalamines or a reaction mixture. Herein, ‘I’ means immediately after the reaction start and ‘C’ means after 2 hours of the reaction. The absorption spectrum of diCNCbl was obtained from the reaction of 20 μM CNCbl with 10 min KCN in 50 mM Tris/HCl (pH 7.5).

FIGS. 2A-2C show a reaction absorption spectrum of 20 μM GSCbl and 10 μM KCN in 50 min Tris/HCl (pH 7.5). CNCbl and diCNCbl are generated in the reaction of GSCbl with cyanide at a low concentration.

FIG. 2A is an absorption spectrum measured at a reaction time of 0 min (GSCbl), 5 min, and 10 min. The graphics is an absorption spectrum obtained by subtracting a spectrum of GSCbl from other spectra

FIG. 2B is an absorption spectrum measured every 5 minute of the reaction after FIG. 2A. The graphics is an absorption spectrum obtained by subtracting a first spectrum of diCNCbl from other spectra. An arrow represents an increase and a reduction of absorption according to a reaction.

FIG. 2C is a graph illustrating a change in absorption at a wavelength indicated according to a reaction time.

FIG. 3A illustrates absorption at 580 nm according to the generation of diCNCbl from the reaction of 50 μM GSCbl with 40 μM KCN in a buffer of pH=4.0 to 12.0. An arrow illustrates an increase of A580 nm at reaction 10 min by an increase of pH in the reaction buffer. (50 min NaCitrate pH 4.0 to 5.0; 50 min MES pH 5.5 to 6.5; 50 min Tris/HCl pH 7.0 to 8.5; 50 min CHES 9.0 to 10.0; 50 min NaHCO₃ pH 10.5 to 11.0; 50 min Na₂HPO₄ pH 11.5 to 12.0)

FIG. 3B illustrates absorption at 361 nm according to the generation of CNCbl from the reaction of 50 μM GSCbl with 40 μM KCN in a buffer of pH=4.0 to 12.0. An arrow illustrates an increase of A361 nm at reaction 120 min by an increase in pH in the reaction buffer. (50 min NaCitrate pH 4.0 to 5.0; 50 min MES pH 5.5 to 6.5; 50 min Tris/HCl pH 7.0 to 8.5; 50 min CHES 9.0 to 10.0; 50 min NaHCO₃ pH 10.5 to 11.0; 50 min Na₂HPO₄ pH 11.5 to 12.0)

FIG. 3C is a graph illustrating a maximum of ΔA580 nm (A580 nm at 10 min-A580 nm at 0 min) plots according to a change of pH. (ΔA=A580 nm at 10 min or A361 nm at 80 min-corresponding absorption at 0 min)

FIG. 3D is a graph illustrating a maximum of ΔA361 nm (A361 nm at 120 min-A361 nm at 0 min) plots according to a change of pH. (ΔA=A580 nm at 10 min or A361 nm at 80 min-corresponding absorption at 0 min)

FIGS. 4A and 4B are results of identifying reaction products CNCbl and GSH of 50 μM GSCbl and 200 μM KCN by HPLC analysis. In FIG. 4A, 20.9 min of a retention time of a cobalamin product is the same as that of CNCbl which is a genuine. In FIG. 4B, 17.5 min of a retention time of a glutathione product is the same as that of GSH which is a genuine.

FIG. 4C is a result of identifying a reaction product GSH of 50 μM GSCbl and 0 to 200 μM KCN by HPLC analysis. A plot of FIG. 4C illustrates a linearly proportional relationship between GSH and KCN. A slope of 0.76±0.04 (n≧3, r²=0.9954) obtained by regression analysis (solid line) of plots corresponds to a molar ratio of [GSH]:[CN⁻]=approximately 1.0:1.3.

FIGS. 5A-5D show anion specificity of diCNCbl generated from GSCbl and a result of naked eye detection of cyanide.

FIG. 5A is an absorption spectrum of 50 μM GSCbl and 5 min CN⁻ or 5 mM other indicated anions in 50 min Tris/HCl (pH 7.5).

FIG. 5B is a photograph identifying solution colors of 50 μM GSCbl and 5 mM CN⁻ or 5 min other indicated anions.

FIG. 5C is a photograph identifying solution colors of 50 μM GSCbl and 5 mM CN⁻ in a presence of 500 min of indicated added anions.

FIG. 5D is a photograph of solution colors identified after the reaction of 10 min at a concentration of 50 μM GSCbl and indicated CN⁻ μM in 50 min CHES (pH 10.0).

FIGS. 6A-6B show a spectrograph titration of CNCbl for quantifying cyanide.

FIG. 6A is an absorption spectrum obtained after 2 hr of the reaction of 50 μM GSCbl and 0 to 200 μM KCN in 50 min Tris/HCl (pH 9.0). Herein, an arrow illustrates that absorption is increased at A361 nm by an increase in a concentration of KCN.

FIG. 6B illustrates a linearly proportional relationship between CNCbl (De361 nm=14.2 mM⁻¹ cm⁻¹, graphics) and CN⁻, as a graph illustrating ΔA361 nm by plots according to a concentration change of KCN. In the graphics, a slope of 0.83±0.09 (n≧6, r²=0.9922) obtained by regression analysis (solid line in the graphics) of plots corresponds to a molar ratio of [CNCbl]:[CN⁻]=approximately 1.0:1.2.

FIG. 7 is a quantified result of cyanide through fluorometric detection of GSH. After 2 hr of the reaction of 20 μM GSCbl and 0 to 50 μM KCN in 50 mM Tris/HCl (pH 9.0), GSH extracted from GSCbl is detected by using a fluororesence reagent (MCB). In the graphics, plots of fluorescence strength according to the KCN concentration illustrate a linearly proportional relationship between GSH and CN⁻. In the graphics, a slope of 0.66±0.05 (n≧6, r²=0.9941) obtained by regression analysis (solid line) of plots corresponds to a molar ratio of [GSH]:[CN⁻]=approximately 1.0:1.5.

FIGS. 8A-8B are photographs of detecting cyanide in water samples by the naked eye.

FIG. 8A is a photograph obtained by adding an indicated concentration (μM) of cyanide to tap water.

FIG. 8B is a photograph obtained by adding an indicated concentration (μM) of cyanide to pond water.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Hereinafter, the present disclosure will be described in detail.

Composition for Detecting Cyanide (CN⁻)

The present disclosure provides a composition for detecting cyanide (CN−) including glutathionylcobalamin (GSCbl) and a buffer.

The glutathionylcobalamin (GSCbl) is one of vitamin B12 derivatives in which glutathione (GSH) is bound to the central cobalt (Co) of cobalamin (Cbl) having a planar structure in an axial direction.

The composition according to the present disclosure has a specificity (see ‘evaluation of anion specificity’ of Experimental Example 1 and Experimental Example 4) in which GSCbl does not react with other anions by nucleophilic substitution, but selectively reacts with only CN⁻ by displacement. Further, GSH bound to Cbl reacts with CN⁻ by nucleophilic substitution with high efficiency to enhance sensitivity, and cyanocobalamin (CNCbl), di-cyanocobalamin (diCNCbl) and glutathione (GSH) which are byproducts generated by nucleophilic substitution reaction of CN⁻ may be qualitatively/quantitatively detected through spectrophotometric, naked eye, and fluorometric assays, respectively.

The inventors of the present disclosure displaced GSH bound to Cbl into other substituents, but in terms of efficiency of nucleophilic substitution reaction with cyanide anion, it was identified that the GSH substituent was significantly excellent as compared with other substituents, and glutathionylcobalamin (GSCbl) was used for detecting cyanide anion.

Herein, CNCbl is one of byproducts generated when GSH bound to the central cobalt (Co) of cobalamin (Cbl) having a planar structure in an axial direction and cyanide anion are generated by nucleophilic substitution reaction, and may be qualitatively or quantitatively detected by using a UV-Vis spectrophotometer.

Further, diCNCbl is one of byproducts generated when cyanide anion is bound to the central cobalt (Co) of cobalamin (Cbl) having the planar structure in the lower and upper axial directions and has a characteristic that diCNCbl is converted into CNCbl due to low stability. However, at pH 9 or higher pH, the stability of diCNCbl is enhanced and thus a time when the structure is maintained is increased (see evaluation of effect of pH value in Experimental Example 1). Since GSCbl represents red diCNCbl represents purple, GSCbl and diCNCbl may be used for naked eye detection.

Furthermore, GSH is a byproduct separated from GSCbl by the nucleophilic substitution reaction with cyanide anion, and GSH is coupled with a fluororesence reagent (monochlorobimane, MCB) by a catalyst action of glutathione S-transferase (GST) to be used in fluorometric detection.

Spectrophotometric Detection Method

The present disclosure provides a spectrophotometric detection method of cyanide including: the steps of adding a sample including cyanide (CN⁻) to a buffer including glutathionylcobalamin (GSCbl) (Step 1);

reacting the cyanide (CN⁻) added in Step 1 with GSH by displacement through nucleophilic attack, in which GSCbl has a characteristic that glutathione (GSH) is bound to the central cobalt (Co) of cobalamin (Cbl) having a planar structure in an axial direction (Step 2); and

qualitatively or quantitatively detecting the CNCbl generated in Step 2 by a UV-Vis spectrophotometer (Step 3).

In the spectrophotometric detection method according to the present disclosure, the buffer may have less than pH 9, preferably. The reason is that at pH 9 or more, the stability of diCNCbl is enhanced and thus an error in the quantitative detection may be reduced (see evaluation of effect of pH value in Experimental Example 1).

In the spectrophotometric detection method according to the present disclosure, the intensity at an absorption peak at 361 nm which is a characteristic of CNCbl may be measured and quantified, and the quantity of the cyanide anions may be calculated by using the intensity.

A detection limit of the spectrophotometric detection method according to the present disclosure is 1.0 μM and the sensitivity is very high (see ‘quantity of cyanide by spectrophotometric detection of CNCbl’ in Experimental Example 1).

Naked Eye Detection Method

The present disclosure provides a naked eye detection method of cyanide including: the steps of adding a sample including cyanide (CN⁻) to a buffer including glutathionylcobalamin (GSCbl) (Step 1);

reacting the cyanide (CN⁻) added in Step 1 with GSH by displacement through nucleophilic attack, in which GSCbl has a characteristic that glutathione (GSH) is bound to the central cobalt (Co) of cobalamin (Cbl) having a planar structure in an axial direction (Step 2); and

detecting the diCNCbl generated in Step 2 by the naked eye by a change in color (Step 3).

In the naked eye detection method according to the present disclosure, the buffer may have pH 9 or higher pH, preferably. The reason is that at lower than pH 9, the stability of the diCNCbl is lowered and thus the naked eye detection is difficult (see ‘evaluation of effect of pH value’ in Experimental Example 1).

The naked eye detection method according to the present disclosure can distinguish a color change by diCNCbl (purple) generated by adding the sample including cyanide to GSCbl (red) by the naked eye (see Experimental Example 4) to be applied to a rapid inspection kit.

Kit for Detecting Cyanide (CN⁻)

The present disclosure provides a kit for detecting cyanide (CN⁻) in water, blood or food samples comprising the composition for detecting cyanide (CN⁻) having glutathionylcobalamin (GSCbl) and the buffer.

The kit according to the present disclosure uses a principle of the naked eye detection method of the cyanide and may be manufactured by a general method for manufacturing the kit.

Fluorometric Detection Method

The present disclosure provides a fluorometric detection method of cyanide including: the steps of adding a sample including cyanide (CN⁻) to a buffer including glutathionylcobalamin (GSCbl) (Step 1);

reacting the cyanide (CN⁻) added in Step 1 with GSH by displacement through nucleophilic attack, in which GSCbl has a characteristic that glutathione (GSH) is bound to the central cobalt (Co) of cobalamin (Cbl) having a planar structure in an axial direction (Step 2); and

qualitatively or quantitatively detecting the GSH generated in Step 2 by a fluorometric detector (Step 3).

In the fluorometric detection method according to the present disclosure, in the buffer in Step 1, a fluororesence reagent (for example, monochlorobimane (MCB) and the like) coupled with GSH is included and glutathione S-transferase which helps GSH to be coupled with the fluororesence reagent may be further included.

In the fluorometric detection method according to the present disclosure, the fluorescent intensity of GSH may be measured and quantified, and the quantity of the cyanide anions may be calculated by using the fluorescent intensity of GSH.

A detection limit of the fluorometric detection method according to the present disclosure is 1.2 μM and the sensitivity is very high (see Experimental Example 5).

Hereinafter, the present disclosure will be described in more detail by Examples. However, the following Examples just exemplify the present disclosure, and the contents of the present disclosure are not limited to the following Examples.

Materials

Chemicals were purchased from Sigma. The glutathionylcobalamin (GSCbl) was synthesized in the reaction of aquocobalamin (OH₂Cbl) with glutathione (GSH) which is known in the related art with extensive washing to remove free GSH. The concentrations of synthesized GSCbl were determined based on ε534 nm=7.97 mM⁻¹ cm⁻¹. A potassium cyanide (KCN) solution was dissolved in a 20 min sodium hydroxide aqueous solution to be prepared before use and cyanide standard purchased from Sigma used for a quality control of the determination methods. Chloro-2,4-dinitrobenzene (CDNB) and monochlorobimane (MCB) were freshly dissolved in methanol for use.

### <Experimental Example 1> Reaction of Glutathionylcobalamin (GSCbl) with Cyan and Spectrophotometric Determination of Cyanide

In the reaction mixture, 50 μM GSCbl was included in 50 min Tris/HCl pH 7.5 and the indicated concentrations of KCN was added to initiate the reaction. The reaction mixture was incubated in the dark at room temperature and the absorption was measured according to a reaction time (Cary 100 UV-Vis spectrophotometer (Varion)). The final reaction product CNCbl was incubated after 2 hr by adding 50 μM GSCbl and KCN 0 to 200 μM to 50 min Tris/HCl pH 9.0 and then titrated by measuring the absorption (Δε361 nm=14.2 mM⁻¹ cm⁻¹). The molecular ratio of CNCbl and CN was estimated from a simple linear regression analysis of ΔA361 nm and a KCN concentration graph. The cyanide detection limit was calculated according to the IUPAC recommendation. The lowest limit of detection was 3 SD_{b}/s, and herein, SD_{b} is the standard deviation of the blank measurements (n≧7) and s is the slope of the titration curve.

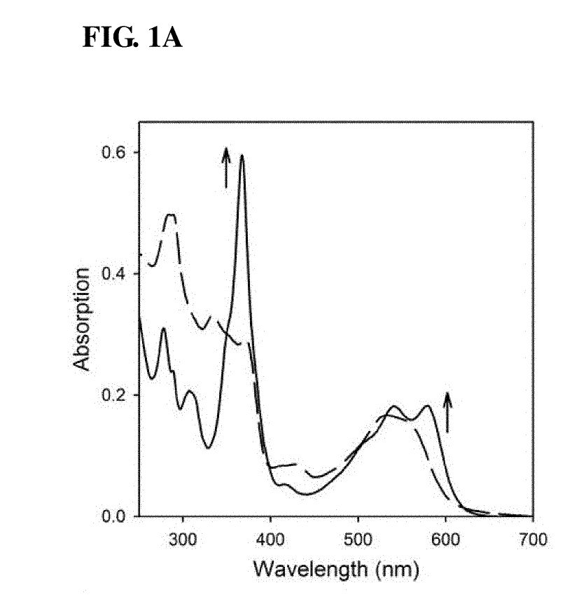

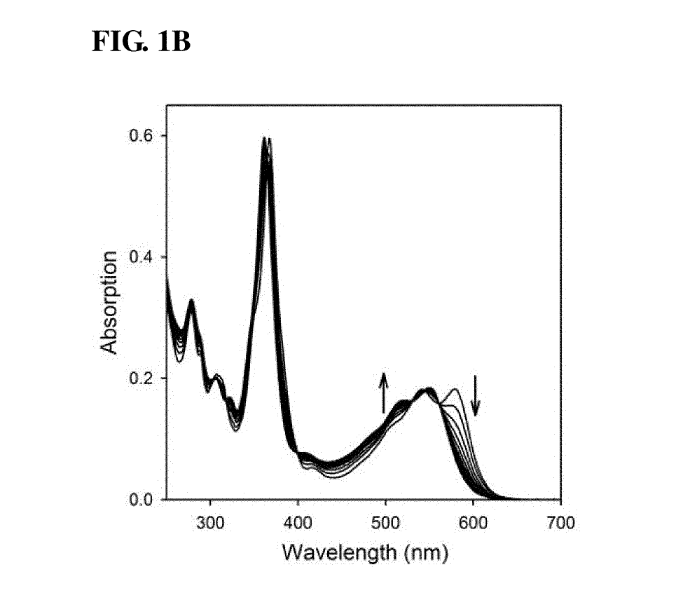

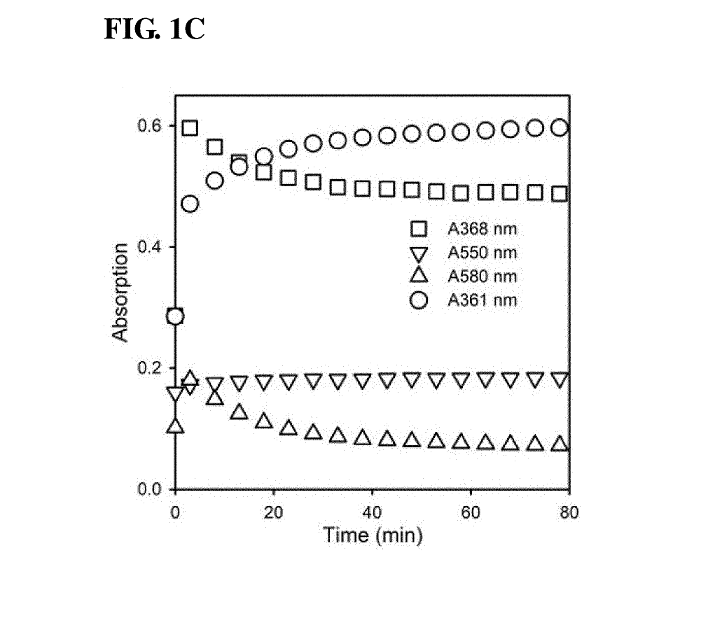

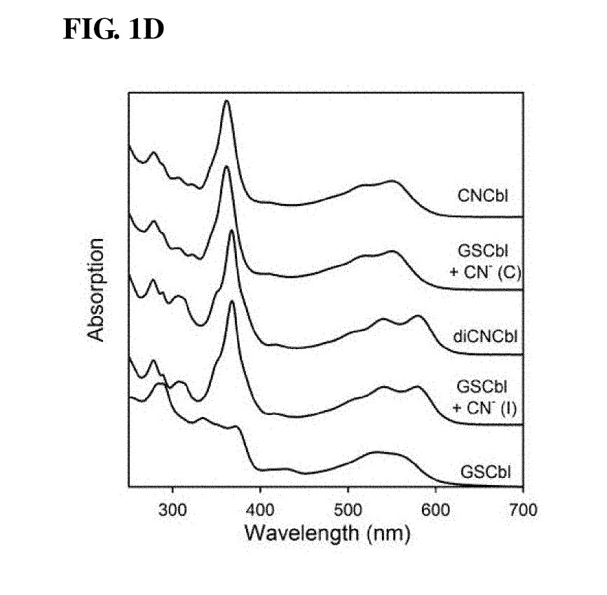

FIG. 1 is a reaction absorption spectrum of 20 μM GSCbl and 1 min KCN in 50 min Tris/HCl (pH 7.5). CNCbl is generated through the intermediate diCNCbl in the reaction of excess cyanide with GSCbl.

FIG. 1A is an absorption spectrum of GSCbl (dotted line) and a reaction (solid line) immediately after a start of the reaction.

FIG. 1B is an absorption spectrum measured every 5 minute of the reaction after FIG. 1A. An arrow represents an increase and a reduction of absorption according to a reaction.

FIG. 1C is a graph illustrating a change in absorption at a wavelength indicated according to a reaction time.

FIG. 1D is a graph illustrating a comparison of absorption spectrums of the indicated cobalamines or a reaction mixture. Herein, ‘I’ means immediately after the reaction start and ‘C’ means after 2 hours of the reaction. The absorption spectrum of diCNCbl was obtained from the reaction of 20 μM CNCbl with 10 min KCN in 50 mM Tris/HCl (pH 7.5).

As illustrated in FIG. 1, when excess cyanide was added in GSCbl, as shown in development of absorption peaks at 368 nm, 541 nm, 580 nm (see FIGS. 1A and 1C) as a characteristic (see FIG. 1D) of di-cyanocobalamin (diCNCbl), the absorption spectrum was immediately and noticeably changed. When the reaction further progressed, in the absorption spectrum of the diCNCbl, an additional slow change was shown, in which the absorption decreased at 368 nm, 541 nm, and 580 nm and the absorption increased at 361 nm, 518 nm, and 550 nm.

Further, in the slow reaction phase, isosbestic points were observed at 364 nm, 399 nm, and 560 nm (see FIGS. 1B and 1C).

The generated absorption spectrum was characteristic of cyanocobalamin (CNCbl) (see FIG. 1D).

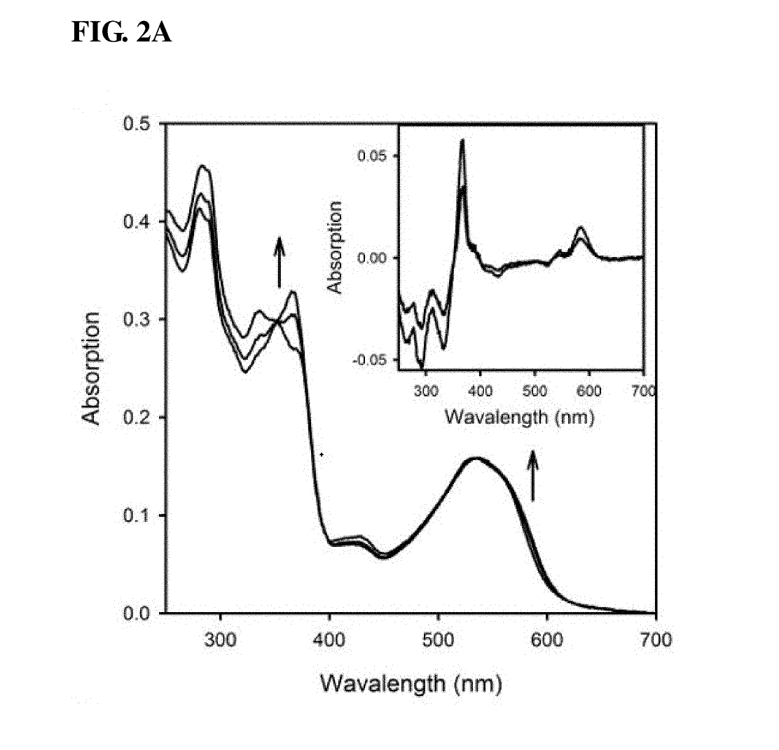

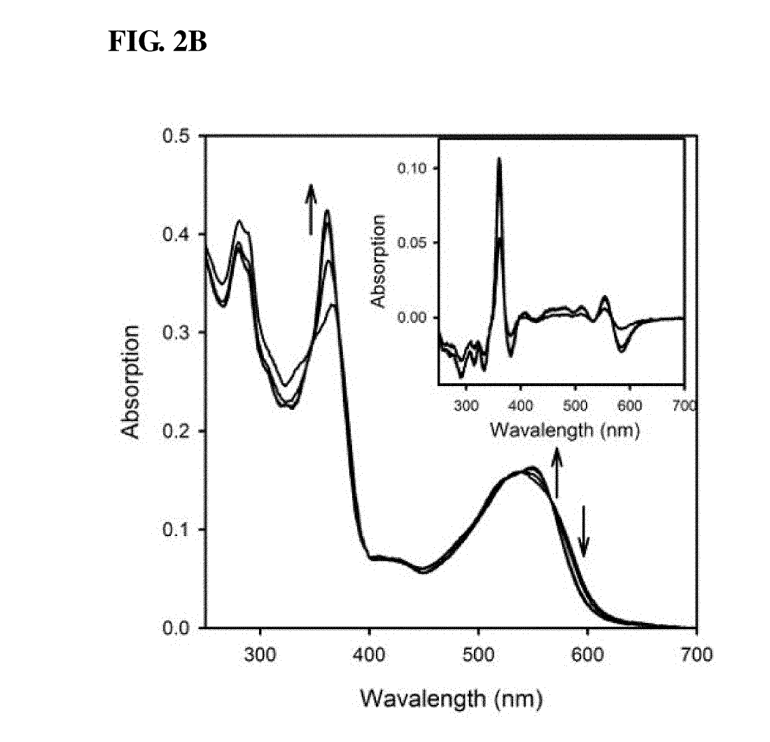

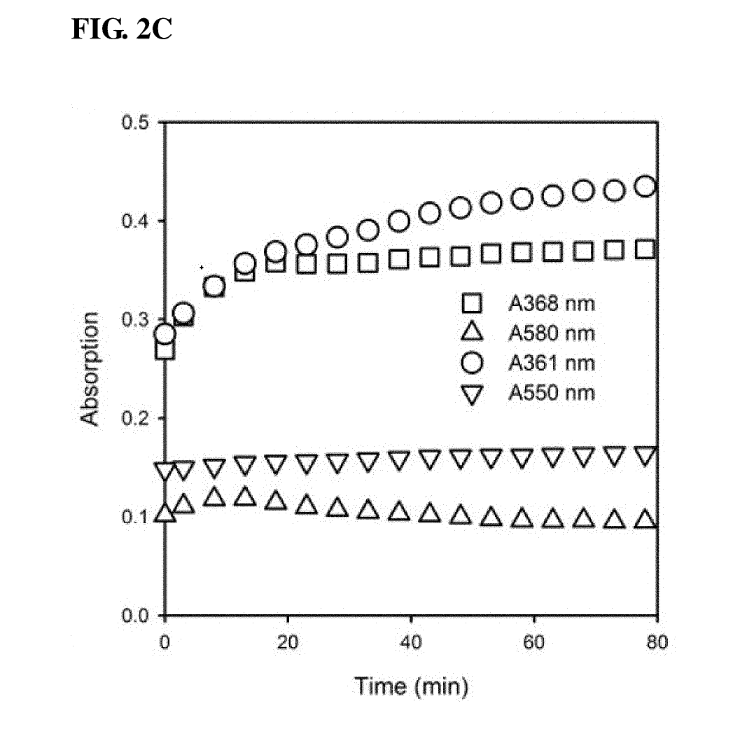

FIG. 2 is a reaction absorption spectrum of 20 μM GSCbl and 10 μM KCN in 50 min Tris/HCl (pH 7.5). CNCbl and diCNCbl are generated in the reaction of GSCbl with low concentrations of cyanide.

FIG. 2A is an absorption spectrum measured at a reaction time of 0 min (GSCbl), 5 min, and 10 min. The graphics is an absorption spectrum obtained by subtracting a spectrum of GSCbl from other spectra.

FIG. 2B is an absorption spectrum measured every 5 minute of the reaction after FIG. 2A. The graphics is an absorption spectrum obtained by subtracting a first spectrum of diCNCbl from other spectra. An arrow represents an increase and a reduction of absorption according to a reaction.

FIG. 2C is a graph illustrating a change in absorption at a wavelength indicated according to a reaction time.

As illustrated in FIG. 2, similar absorption spectrum changes was observed for the reaction of GSCbl with low concentrations of cyanide reaction with GSCbl at a ratio of [GSCbl]/[cyanide]=2.0 (see FIG. 2). In a fast reaction phase, absorption peaks at 368 nm and 580 nm were developed, which was characteristic of the generation of diCNCbl although to a lower extent than that obtained with excess cyanide (see FIG. 2A). In the later slow reaction phase, the absorption peaks at 368 nm and 580 nm decreased with concomitant absorption increases at 361 nm and 550 nm (see FIG. 2C), which was the characteristic shown when the diCNCbl was converted into the CNCbl. The results showed that the cyanide reacts with the GSCbl and displaces the GSH ligand to generate the CNCbl via a diCNCbl intermediate.

Evaluation of Effect of pH Value

Induction of reaction of cyanide anions by nucleophilic attack

At a ratio of [GSCbl]/[cyanide]=0.8, concentrations of GSCbl and cyanide were fixed and in a range of pH of 4.0 to 12.0, an effect of pH at the reaction of the GSCbl and the cyanide was evaluated.

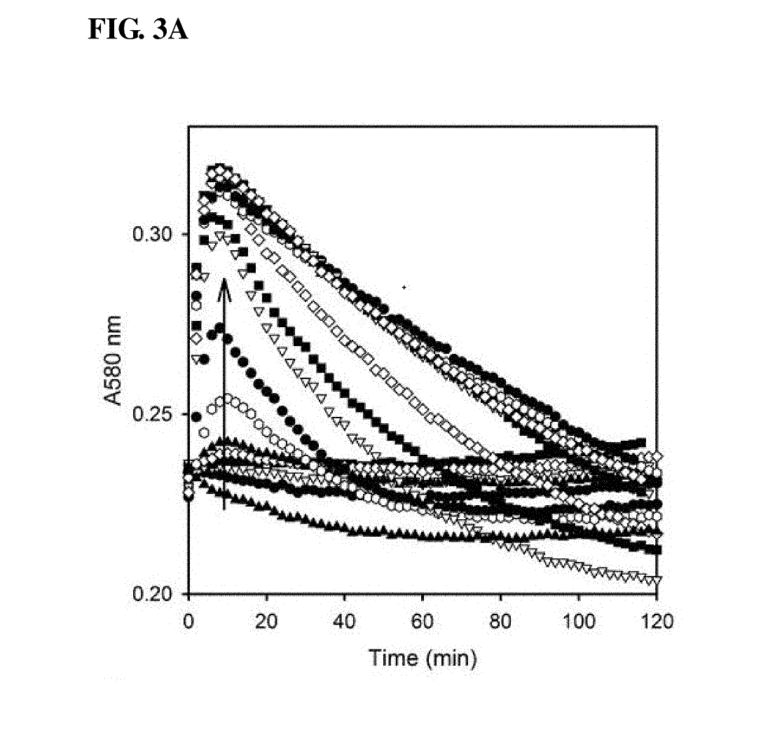

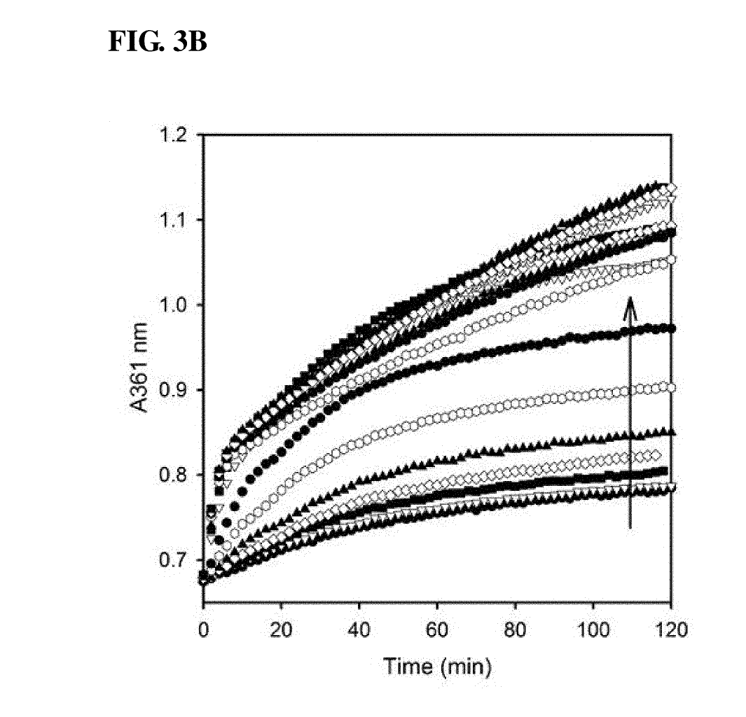

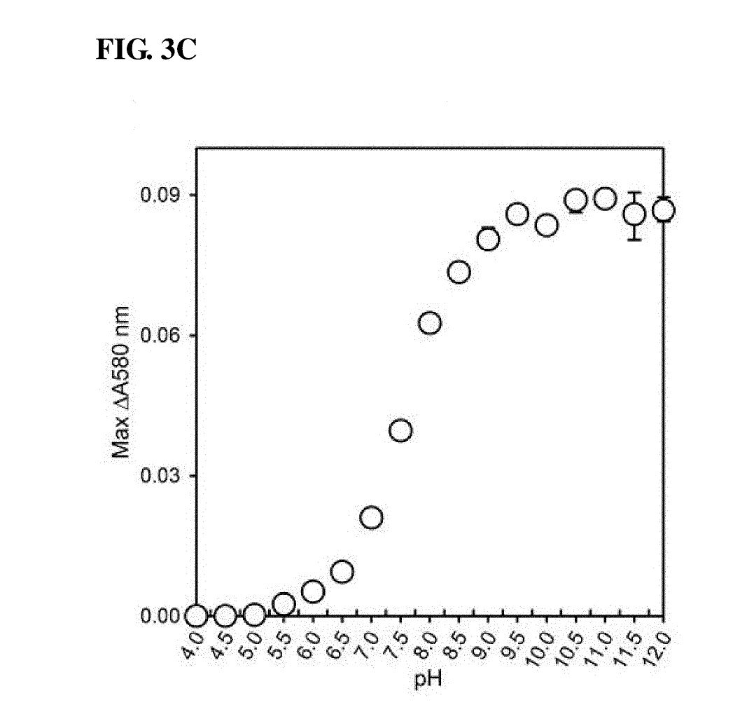

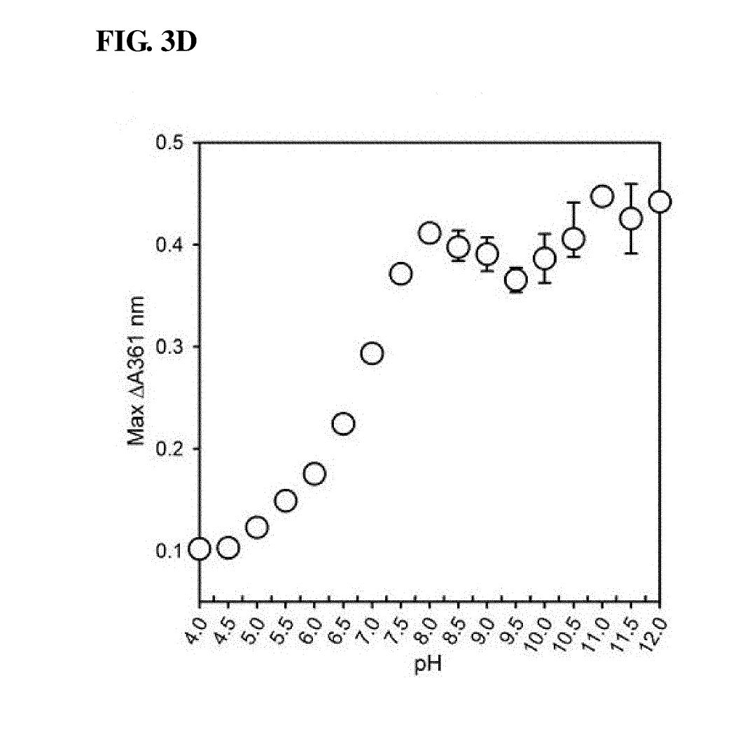

FIG. 3A illustrates absorption at 580 nm according to generation of diCNCbl from the reaction of 50 μM GSCbl with 40 μM KCN in a buffer of pH=4.0 to 12.0. An arrow illustrates an increase of A580 nm at reaction 10 min by increasing pH of the reaction buffer. (50 min NaCitrate pH 4.0 to 5.0; 50 min MES pH 5.5 to 6.5; 50 mM Tris/HCl pH 7.0 to 8.5; 50 min CHES 9.0 to 10.0; 50 min NaHCO₃ pH 10.5 to 11.0; 50 mM Na₂HPO₄ pH 11.5 to 12.0)

FIG. 3B illustrates absorption at 361 nm according to the generation of CNCbl from the reaction of 50 μM GSCbl with 40 μM KCN in a buffer of pH=4.0 to 12.0. An arrow illustrates an increase of A361 nm at reaction 120 min by an increase in pH in the reaction buffer. (50 min NaCitrate pH 4.0 to 5.0; 50 min MES pH 5.5 to 6.5; 50 min Tris/HCl pH 7.0 to 8.5; 50 min CHES 9.0 to 10.0; 50 min NaHCO₃ pH 10.5 to 11.0; 50 min Na₂HPO₄ pH 11.5 to 12.0)

FIG. 3C is a graph illustrating a maximum of ΔA580 nm (A580 nm at 10 min-A580 nm at 0 min) plots according to a change of pH. (ΔA=A580 nm at 10 min or A361 nm at 80 min-corresponding absorption at 0 min)

FIG. 3D is a graph illustrating a maximum of ΔA361 nm (A361 nm at 120 mM-A361 nm at 0 min) plots according to a change of pH. (ΔA=A580 nm at 10 min or A361 nm at 80 min-corresponding absorption at 0 min)

As illustrated in FIG. 3, the generation of the reaction intermediate diCNCbl and the reaction product CNCbl was followed by measuring the A580 nm and A361 nm, respectively (see FIGS. 3A and 3B). The absorption at 580 nm increased for 10 mM and then decreased for an additional reaction time (see FIG. 3A). The result means the rapid generation of diCNCbl and disappearance thereof.

A diCNCbl maximum value was increased by an increase of pH at 10 min (see FIG. 3C). It was evident that the pH optimal range for the generation of diCNCbl was pH >9.0. The result shown that the reaction of GSCbl with the cyanide is induced by the nucleophilic attack of the cyanide anion. After the reaction, the absorption at 361 nm increased and then reached equilibrium, and the result means the saturation of the generation of CNCbl.

The levels of CNCbl at 2 hr were increased according to the increase of pH (see FIG. 3D). It was shown that the generation of CNCbl was optimized in the range of pH ≧8.0. However, at pH=8.5, small interference in the absorption at 361 nm occurred, which due to the enhanced stability of diCNCbl and its incomplete conversion into CNCbl.

Evaluation of Anion Specificity for Displacement of Glutathione Ligand of GSCbl

In order to experiment anion specificity for the reaction of GSCbl with CN⁻, various anions (Cl⁻, F⁻, Br⁻, SCN⁻, NO₃⁻, HCO₃⁻, PO₄³⁻, or SO₄²⁻) reacted with the GSCbl and then an absorption spectrum was measured.

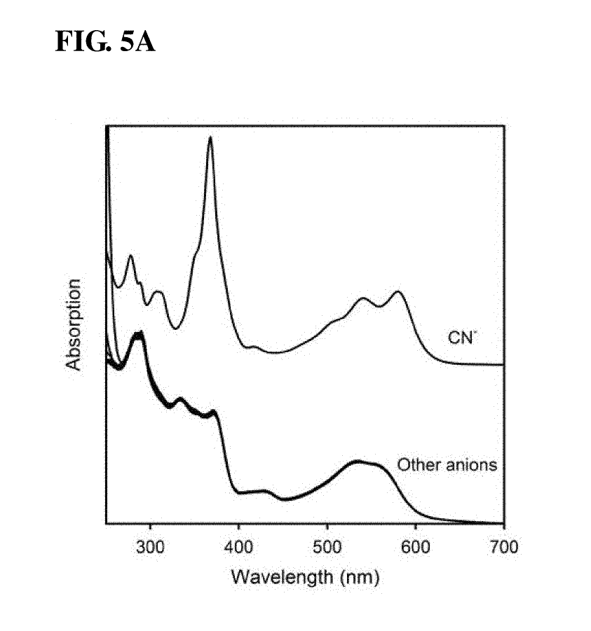

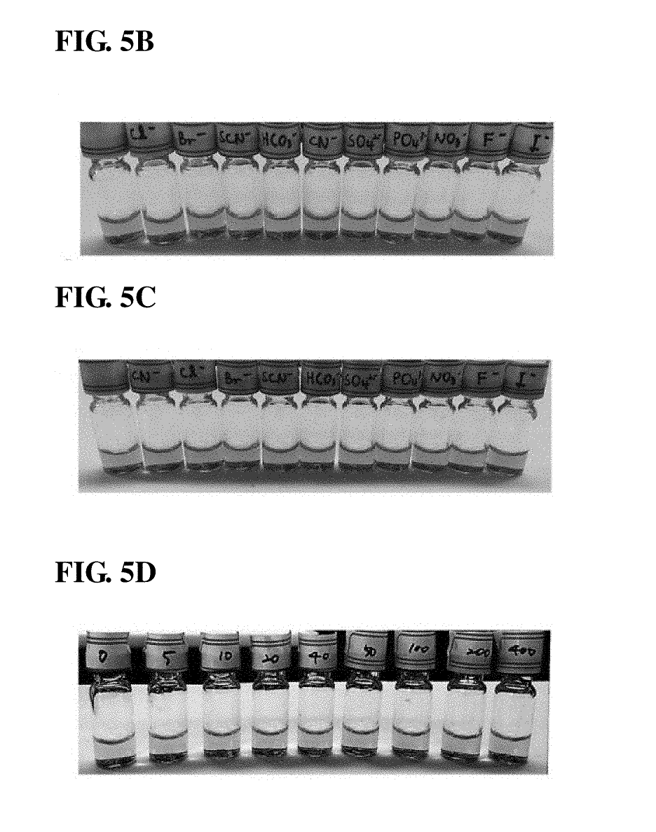

FIG. 5 illustrates anion specificity of diCNCbl generated from GSCbl and a result of naked eye detection of cyanide.

FIG. 5A is an absorption spectrum of 50 μM GSCbl and 5 min CN⁻ or 5 mM other indicated anions in 50 min Tris/HCl (pH 7.5).

As illustrated in FIG. 5A, characteristic absorption bands of GSCbl (λmax=288 nm, 334 nm, 428 nm, and 534 nm) remained unaffected, and the result indicated that no glutathione ligand displacement occurred (see FIG. 5A). However, the addition of excess cyanide (CN⁻) induced an immediate change of the absorption spectrum which was shown in the absorption peaks of 368 nm, 541 nm, and 580 nm as the characteristic of the diCNCbl. Furthermore, in the presence of excess other anions, at 100-fold excess over CN⁻, the addition of CN⁻ to GSCbl showed the same absorption spectral changes of diCNCbl generation. The results indicate that the displacement of the glutathione ligand of GSCbl is specific for the cyanide anion without competition against any of other tested anions.

Quantitation of Cyanide by Spectrophotometric Determination of CNCbl

The generation of CNCbl in the reaction of GSCbl with increasing concentrations of cyanide was measured by a UV-Vis spectroscopy.

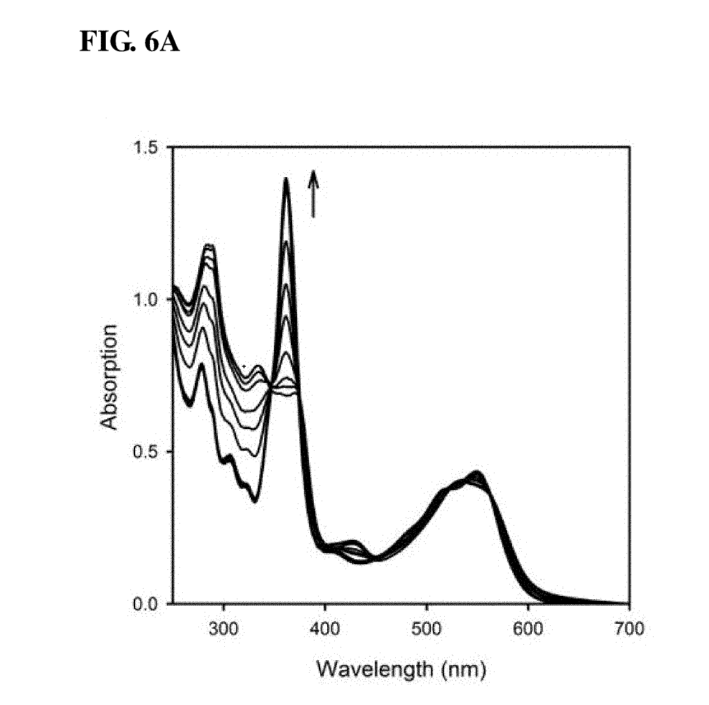

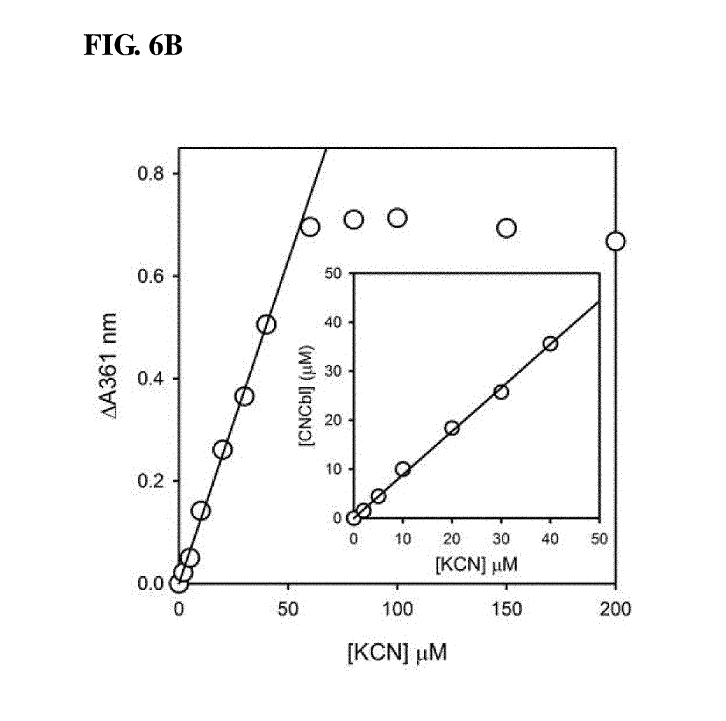

FIG. 6 is a spectrograph titration of CNCbl for quantifying cyanide.

FIG. 6A is an absorption spectrum obtained after 2 hr of the reaction of 50 μM GSCbl with 0 to 200 μM KCN in 50 min Tris/HCl (pH 9.0). Herein, an arrow illustrates that absorption is increased at A361 nm by an increase in a concentration of KCN.

FIG. 6B illustrates a linearly proportional relationship between CNCbl (De361 nm=14.2 mM⁻¹ cm⁻¹, graphics) and CN⁻, as a graph illustrating ΔA361 nm by plots according to a concentration change of KCN. In the graphics, a slope of 0.83±0.09 (n≧6, r²=0.9922) obtained by regression analysis (solid line in the graphics) of plots corresponds to a molar ratio of [CNCbl]:[CN⁻]=approximately 1.0:1.2.

As illustrated in FIG. 6, the absorption spectra of the reaction product showed the increase of CNCbl in response to increasing the concentration of cyanide (see FIG. 6A). On the contrary to the cyanide concentration, the plot of A361 nm revealed a linearly proportional relationship between CNCbl and CN⁻ in the cyanide concentration range of 0 to 50 μM (see FIG. 6B) and a molar ratio of [CNCbl]:[CN⁻] is expected to approximately 1.0:1.2 (see graphics of FIG. 6B). The spectrometric titration may be applied for cyanide quantitation with a detection limit of 1.0 μM cyanide concentration.

### <Experimental Example 2> HPLC Analysis

A cobalamin product was identified by the HPLC analysis according to a known method. A reaction mixture was prepared with 50 μM GSCbl and 200 μM KCN in 50 min Tris/HCl pH 7.5 and incubated for 2 hr in the dark at room temperature, and then loaded on an Inersil ODS-3V C₁₈ reversed phase column (250×4.6 mm, 5 μm, GL Sciences). The column was then eluted with a gradient ranging from 0% to 40% acetonitrile in 0.1% TFA aqueous solution for 40 min at a flow rate of 1 ml min′ and the absorption at 254 nm was measured. Under these conditions, standard cobalamin OH₂Cbl, CNCbl, and GSCbl was eluted at retention times of 17.6 min, 20.9 min, and 22.6 min, respectively. The retention time of the cobalamin product from the reaction of GSCbl and KCN was compared with the retention times of the standard cobalamins.

The glutathione product was identified and quantified by HPLC analysis as previously described. The reaction mixtures were prepared with 50 μM GSCbl and 0 to 200 μM KCN in 50 min Tris/HCl pH 7.5 and incubated for 2 hr in the dark at room temperature. Amino groups of glutathione were derivatised with 2,3-dinitrofluorobenzene following the reaction of free thiols with monoiodoacetic acid and injected to a Bondclone NH₂ column (300 mm×3.9 mm, 10 μm, Phenomenex) equilibrated with solvent A of 4:1 (v/v) methanol/water. The column was eluted using a solvent B (the mixture of 400 ml of solvent A with a 100 ml solution of 272 g sodium acetate trihydrate, 122 ml of water, and 373 ml of glacial acetic acid) under the following condition: from 0-5 min, isocratic 30% solvent B; from 5-30 min, linear gradient from 30-100% solvent B. The elution peak of GSH were monitored by measuring the absorption at 355 nm. The retention time of the glutathione product from the reaction of GSCbl with KCN was compared with the retention times of standard GSH and GSSG. The concentrations of the GSH product were determined by comparing integrated peak areas with the standard curve obtained using commercial GSH compound. The stoichiometry of GSH:CN—was estimated from the plot of GSH concentrations versus KCN concentrations by a simple linear regression analysis.

The products from the reaction of GSCbl with cyanide were identified by HPLC analysis.

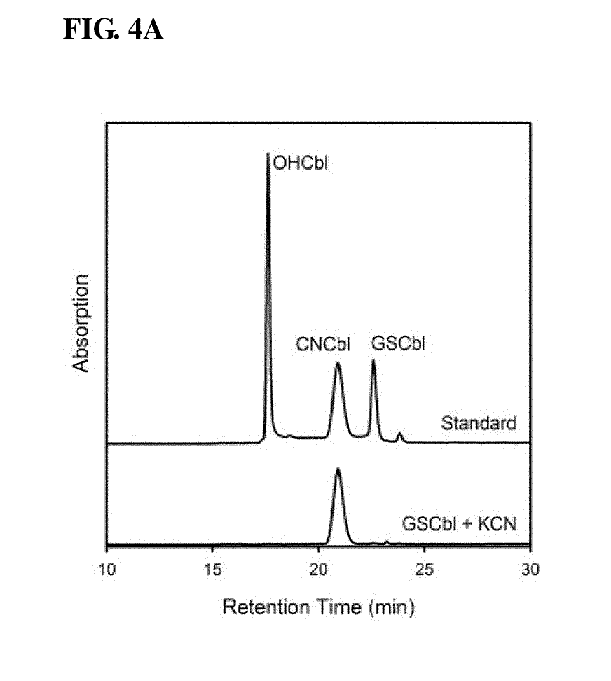

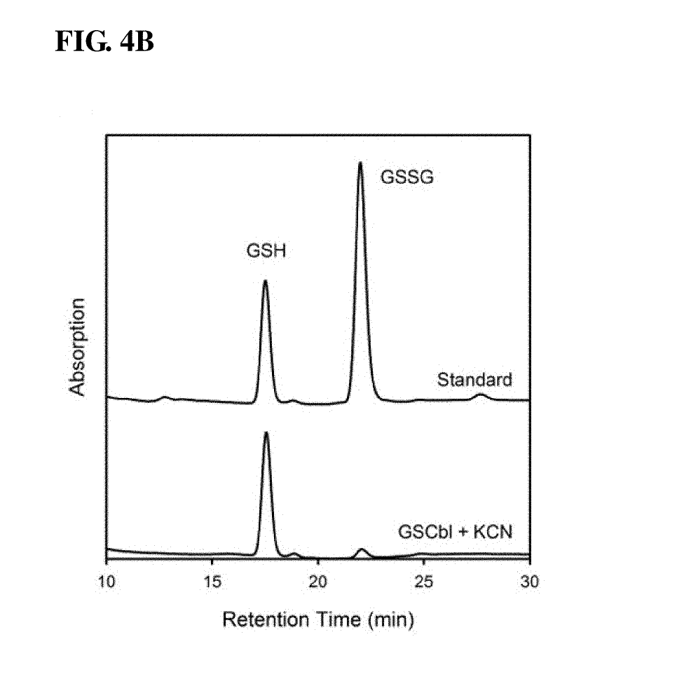

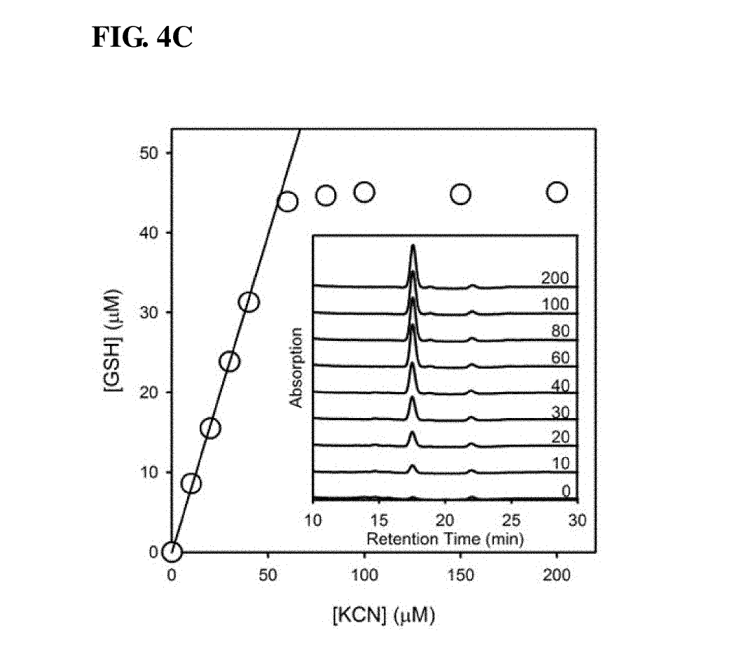

FIGS. 4A and 4B are results of identifying products from the reaction of CNCbl with GSH of 50 μM GSCbl and 200 μM KCN by HPLC analysis. In FIG. 4A, the cobalamin product was identified as CNCbl being eluted at the same retention time of 20.9 min. In FIG. 4B, the glutathione product was in the reduced from, GSH being eluted at the same retention time of 17.5 min.

FIG. 4C is a result of identifying a reaction product GSH of 50 μM GSCbl and 0 to 200 μM KCN by HPLC analysis. A plot of FIG. 4C illustrates a linearly proportional relationship between GSH and KCN. A slope of 0.76±0.04 (n≧3, r²=0.9954) obtained by regression analysis (solid line) of plots corresponds to the molar ratio of [GSH]:[CN⁻]=approximately 1.0:1.3.

As illustrated in FIG. 4, as expected in the absorption spectrum (see FIGS. 1C and 1D) for the reaction of GSCbl with cyanide, the cobalamin product was identified as CNCbl (see FIG. 4A). The other reaction product was assumed to be glutathione (GSH) released from GSCbl by cyanide displacement. The HPLC analysis revealed that the glutathione product was converted from the glutathione (GSH) (see FIG. 4B). Additionally, the quantitative analysis showed a linearly proportional relationship between GSH and CN⁻, and a molar ratio of [GSH]:[CN⁻] was approximately 1.0:1.3 (see FIG. 4C).

### <Experimental Example 3> Synthesis and Isolation of Glutathione S-Transferase

Glutathione S-transferase (GST) from Schistosoma japonicum was prepared by overexpression of the encoding gene in an expression vector pGEX-4T3 (GE Healthcare). E. coli BL21 (DE3) (Novagen) harbouring the pGEX-4T3 plasmid was grown and pre-incubated in a LB medium/ampicillin of 100 g/mL at 37° C. overnight. The main culture of 1 L LB/ampicillin (100 μg/mL) was inoculated with 1% pre-culture and incubated at 37° C. until A600 nm reached approximately 0.8. The gene expression was induced by adding 50 min isopropyl b-d-thiogalactopyranoside (IPTG, Qiagen) and E. coli was incubated for 5 hr at 37° C., and then cells were harvested and lysed for protein purification. The GST was purified by an affinity column chromatography using a GSH sepharose 4b affinity column (5 mL column volume, GE Healthcare) following manufacturer's instruction. The purified GST was extensively dialysed in PBS to remove GSH contaminated during affinity purification. The protein concentrations were determined by a Bradford assay and the GST activity was determined spectrophotometrically using CDNB (Δε340 nm=9.6 mM⁻¹ cm⁻¹).

### <Experimental Example 4> Naked Eye Detection of Cyanide

The reaction of GSCbl with cyanide gemerated the intermediate diCNCbl accompanied by a large bathochromic shift (see FIG. 1A) that could be used for the detection of cyanide by the naked eye.

FIG. 5 illustrates anion specificity of diCNCbl generated from GSCbl and a result of naked eye detection of cyanide.

FIG. 5B is a photograph identifying solution colors of 50 μM GSCbl and 5 mM CN⁻ or 5 min other indicated anions.

FIG. 5C is a photograph identifying solution colors of 50 μM GSCbl and 5 mM CN⁻ in a presence of 500 min of indicated added anions.

FIG. 5D is a photograph of solution colors identified after a reaction of 10 min at a concentration of 50 μM GSCbl and indicated CN⁻ μM in 50 min CHES (pH 10.0).

As illustrated in FIGS. 5B to 5D, when excess CN⁻ was added to the GSCbl, the diCNCbl was generated, resulting in an immediate color change (from red to purple), whereas other anions (5 min each of Cl⁻, F⁻, Br⁻, I⁻, SCN⁻, NO₃⁻, HCO₃⁻, PO₄³⁻, or SO₄²⁻) did not detect the color change (see FIG. 5B). Furthermore, after CN⁻ was added in a container with other anions tested with the GSCbl solution, the colors were changed to purple (see FIG. 5C), and the result also means that the displacement of the glutathione ligand of GSCbl does not compete with other tested anions but specifically reacts with only the cyanide anion. Under the optimized conditions (20 μM GSCbl was incubated for 10 min in a buffer of pH=9.0 to 12.0), the GSCbl may be applied for the naked eye detection of cyanide with an apparent detection limit of approximately 20 μM (see FIG. 5D).

### <Experimental Example 5> Quantitation of Cyanide by GSH Fluorometric Detection of GSH

The reaction mixture was prepared by adding 20 μM GSCbl and 0 to 20 μM KCN in 50 min Tris/HCl pH 9.0 and incubated for 2 hr in the dark at room temperature. The pH of the reaction mixture was adjusted to 7.5 by adding HCl and thereafter, a fluororesence reagent monochlorobimane (MCB, 100 μM) and 1 U/ml GST was added. After the reaction mixture was incubated for 20 min at room temperature, the fluorescence was measured; Synergy HT microplate reader (BioTek), exciting wavelength of 360±40 nm, emission wavelength of 460±40 nm. The GSH concentration was measured by comparing standard curves obtained from a mixture of 20 μM GSCbl and the standard GSH the above method. A detection limit was calculated according to the IUPAC recommendations. The lowest limit of the detection was 3 SD_{b}/s, and herein, SD_{b} was the standard deviation of the blank measured value (n≧7) and s was a slope of the standard curve.

GSH released from the GSCbl by the cyanide displacement was determined to be linearly proportional, therefore, it could be used for cyanide quantitation by the HPLC analysis (see FIG. 4B). However, for simplicity with high sensitivity, in the present disclosure, a thiol-specific fluorescence reagent (monochlorobimane, MCB) was used for GSH determination.

Particularly, the GSCbl was incubated with different concentrations of cyanide and the GSH released from the GSCbl was conjugated with the fluorescence reagent (monochlorobimane, MCB) by catalysis of glutathione S-transferase (GST).

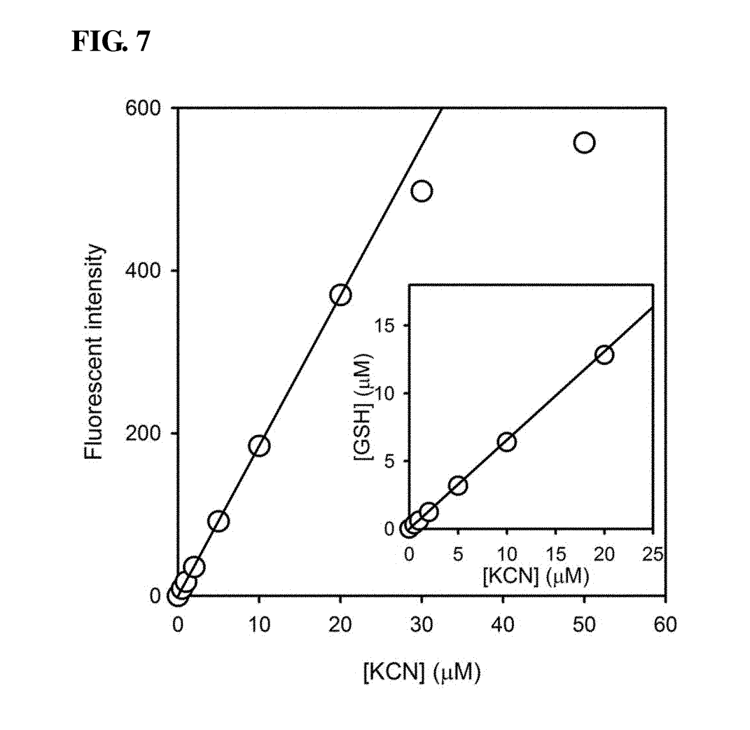

FIG. 7 is a quantified result of cyanide through fluorometric detection of GSH. After 2 hr of the reaction of 20 μM GSCbl and 0 to 50 μM KCN in 50 mM Tris/HCl (pH 9.0), GSH extracted from GSCbl is detected by the fluorescence reagent (MCB). In the graphics, plots of fluorescence intensities measured for the determination of GSH were linearly proportional to CN⁻ in the cyanide concentration range. In the graphics, a slope of 0.66±0.05 (n≧6, r²=0.9941) obtained by regression analysis (solid line) of plots corresponds to a molar ratio of [GSH]:[CN⁻]=approximately 1.0:1.5.

As illustrated in FIG. 7, as a result of measuring the fluorescent intensity for GSH detection, the fluorescent intensity was shown with linear proportion in a range of 0 to 20 μM of the cyanide (CN⁻) concentration and a molar ratio of [GSH]:[CN⁻] was estimated as approximately 1.0:1.5.

The fluorometric detection of the GSH according to the present experiment may be applied to the cyanide quantitation at a detection limit concentration of 1.2 μM.

### <Experimental Example 6> Application in Water Samples

In an actual sample, in order to determine whether GSCbl-based cyanide detection according to the present disclosure can be applied, the experiment was performed by adding cyanide to tap water and pond water.

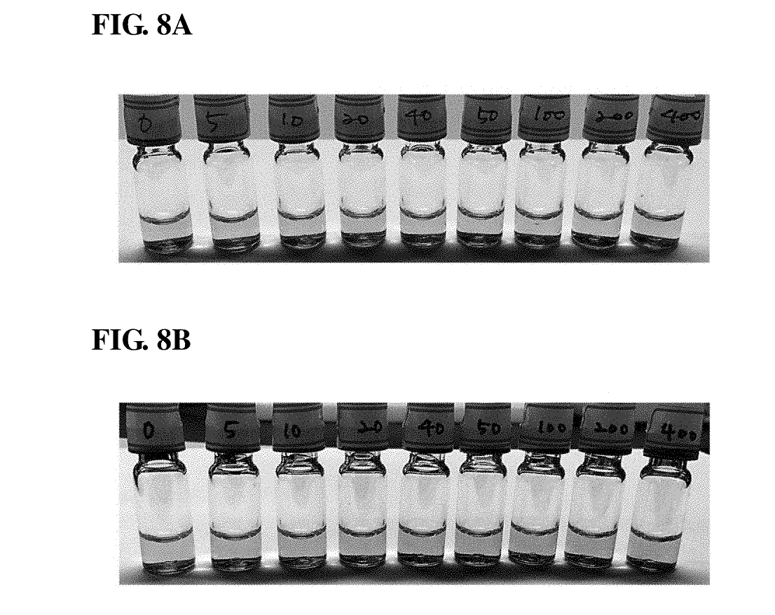

FIG. 8 is a photograph of detecting cyanide in water samples by the naked eye.

FIG. 8A is a photograph obtained by adding a indicated concentration (μM) of cyanide to tap water.

FIG. 8B is a photograph obtained by adding an indicated concentration (μM) of cyanide to pond water.

As illustrated in FIG. 8, the naked eye detection consistently yielded a detection limit of 20 μM cyanide in water samples. Results obtained by spectrophotometric assay and fluorometric assay were in good agreement with the amounts of cyanide added in water samples.

In the following Table 1, a result of detection a concentration of cyanide in a water sample through spectrophotometric and fluorometric evaluation is listed.

TABLE 1

| | Tap water | | Pond water | |
|---|---|---|---|---|
| Added | Spectrophotometric | Fluorometric | Spectrophotometric | Fluorometric |
| concentration | detection | detection | detection | detection |
| of cyanide | concentration | concentration | concentration | concentration |
| (μM) | (μM) | (μM) | (μM) | (μM) |
| 2 | 2.0 ± 0.1 | 2.1 ± 0.3 | 1.7 ± 0.1 | 2.0 ± 0.3 |
| 5 | 5.1 ± 0.1 | 5.1 ± 0.4 | 3.7 ± 0.5 | 5.0 ± 0.4 |
| 10 | 11.5 ± 0.4 | 10.1 ± 0.8 | 9.1 ± 0.3 | 8.9 ± 0.4 |

As listed in Table 1, it can be seen that both a spectrophotometric detection method and a fluorometric detection method can quantitatively detect the cyanide anions with high sensitivity.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A composition for detecting cyanide (CN⁻), the composition comprising glutathionylcobalamin (GSCbl) and a buffer.

2. A spectrophotometric detection method of cyanide, the method comprising:
adding a sample including cyanide (CN⁻) to a buffer including glutathionylcobalamin (GSCbl) (Step 1);
reacting the cyanide (CN⁻) added in Step 1 with GSH by displacement through nucleophilic attack, in which GSCbl has a characteristic that glutathione (GSH) is bound to the central cobalt (Co) of cobalamin (Cbl) having a planar structure in an axial direction (Step 2); and
qualitatively or quantitatively detecting the CNCbl generated in Step 2 by a UV-Vis spectrophotometer (Step 3).

3. The spectrophotometric detection method of claim 2, wherein the buffer of Step 1 has pH lower than pH 9.

4. A naked eye detection method of cyanide, the method comprising:
adding a sample including cyanide (CN⁻) to a buffer including glutathionylcobalamin (GSCbl) (Step 1);
reacting the cyanide (CN⁻) added in Step 1 with GSH by displacement through nucleophilic attack in which GSCbl has a characteristic that glutathione (GSH) is bound to the central cobalt (Co) of cobalamin (Cbl) having a planar structure in an axial direction (Step 2); and
detecting di-cyanocobalamin (diCNCbl) generated in Step 2 by the naked eye by a change in color (Step 3).

5. The naked eye detection method of claim 4, wherein the buffer of Step 1 has pH 9 or higher pH.

6. A kit for detecting cyanide (CN⁻) in a water, blood or food samples comprising the composition of claim 1.

7. A fluorometric detection method of cyanide, comprising the steps of:
adding a sample including cyanide (CN⁻) to a buffer including glutathionylcobalamin (GSCbl) (Step 1);
reacting the cyanide (CN⁻) added in Step 1 with GSH by displacement through nucleophilic attack, in which GSCbl has a characteristic that glutathione (GSH) is bound to the central cobalt (Co) of cobalamin (Cbl) having a planar structure in an axial direction (Step 2); and
qualitatively or quantitatively detecting the GSH generated in Step 2 by a fluorometric detector (Step 3).
